(19) United States
(12) Patent Application Publication
(10) Pub. No.: US 2011/0064777 A1
(43) Pub. Date: Mar. 17, 2011

(54) LIVE LACTOBACILLUS SOAP AND METHOD FOR DELIVERING LACTOBACILLUS TO SKIN TISSUE

(76) Inventor: Sharad Mohan, Takatsuki-Shi (JP)

(21) Appl. No.: 12/878,396

(22) Filed: Sep. 9, 2010

Related U.S. Application Data
(60) Provisional application No. 61/276,462, filed on Sep. 11, 2009.

(51) Int. Cl. A61K 8/99; A61K 8/02; A61Q 19/00

(52) U.S. Cl. 424/401; 424/93.45

(57) ABSTRACT

Microbiotic Soap made with the addition of Lactobacillus or other natural bacteria found in yoghurt. The intention of the invention being to deliver live Lactobacillus and/or other bacteria on to the skin via Microbiotic Soap. Special structures ‘Gel Lactowich’ and ‘Gel Lactospheres’ allow Lactobacillus and other natural bacteria to continue to live in the soap unharmed. On normal use of Microbiotic Soap with water the soap dissolves setting the Lactobacillus free and allowing the Lactobacillus or any other natural bacteria inserted in the soap to attach itself to skin for curing various skin diseases and for cosmetic purposes.

### PRIORITY CLAIM

The present application is a Non-Provisional Utility Patent Application of U.S. Provisional Application No. 61/276,462, filed on Sep. 11, 2009, entitled, “LIVE LACTOBACILLUS SOAP AND METHOD FOR DELIVERING LACTOBACILLUS TO SKIN TISSUE.”

### BACKGROUND OF THE INVENTION

1. Technical Field

The present invention relates to a therapeutic soap and, more particularly to soap having a live Lactobacillus therein for applying to skin tissue and treating dermatological ailments.

2. Description of Related Art

Soaps have commonly been used to cleanse a user's skin tissue. Separately, Lactobacillus bacteria is shown to be of benefit in maintaining good health. However, nothing heretofore devised has combined a skin wash with such a live bacteria. Thus, a continuing need exists for a soap having a Lactobacillus therein.

### SUMMARY OF THE INVENTION

The present invention is a soap with a live Lactobacillus species bacteria positioned within the soap. In other words, the soap is formed in such a manner (as described below) to include live Lactobacillus therein.

The present invention also includes a delivery method, or a method for delivering Lactobacillus onto human or animal skin while using. The method comprises a plurality of steps or acts of forming the soap as described herein. The method also includes steps or acts of applying the soap to the skin tissue, dissolving portions of the soap to release the Lactobacillus, allowing the Lactobacillus and any other natural bacteria (e.g., from yoghurt) to attach itself to the skin tissue for curing various skin diseases and for cosmetic purposes.

For further understanding, the present invention is described in further detail below.

### BRIEF DESCRIPTION OF THE DRAWINGS

The objects, features and advantages of the present invention will be apparent from the following detailed descriptions of the preferred aspect of the invention in conjunction with reference to the following drawings where:

FIG. 1 shows a 2 dimensional side view of a bar of Microbiotic Soap with a Gel Lactowich structure in the middle of the soap having Lactobacillus bacteria at its centre.

FIG. 2 shows a 3 dimensional diagrammatic front and side view of a bar of Microbiotic Soap with a Gel Lactowich structure in the middle of the soap having Lactobacillus bacteria at its centre.

FIG. 3 shows a 2 dimensional side view of a bar of Microbiotic Soap having multiple Gel Lactospheres distributed through out the soap.

FIG. 4 shows a 3 dimensional diagrammatic view of a bar of Microbiotic Soap having multiple Gel Lactospheres distributed through out the soap.

FIG. 5 shows a 3 dimensional diagrammatic view of a bar Microbiotic soap having an alternative/optional structure in which the Gel Lactowich is embedded in the centre of the soap as a ring through out the soap.

### DETAILED DESCRIPTION

The present invention relates to a therapeutic soap and, more particularly to soap having a live Lactobacillus therein for applying to skin tissue and treating dermatological ailments.

The following description, taken in conjunction with the referenced drawings, is presented to enable one of ordinary skill in the art to make and use the invention and to incorporate it in the context of particular applications. Various modifications, as well as a variety of uses in different applications, will be readily apparent to those skilled in the art, and the general principles defined herein, may be applied to a wide range of aspects. Thus, the present invention is not intended to be limited to the aspects presented, but is to be accorded the widest scope consistent with the principles and novel features disclosed herein. Furthermore, it should be noted that unless explicitly stated otherwise, the figures included herein are illustrated diagrammatically and without any specific scale, as they are provided as qualitative illustrations of the concept of the present invention.

As noted herein, the present invention is direct to a Micro-biotic Soap. This invention and discovery relates to an improved and extremely effective way to increase the efficacy of soap and cure dermatological ailments and pathology while increasing the cosmetic benefits of the use of soap.

Soap is a globally accepted way of disinfecting and cleaning ones body and maintaining hygiene. It is also used to prevent infections and sometimes used for cosmetic benefits.

Though soap is being used on a regular basis many dermatological problems including infections and other pathologies are increasing, and normal soap is unable to prevent or cure dermatological problems including infections arising from fungus or bacteria. The cosmetic beneficial effects of ordinary soap on a persons face and body is also negligible or non-existent. To overcome these problems, the current invention and discovery has added Lactobacillus bacteria species from natural yoghurt to ordinary soap in a highly specialized manner as to allow the live Lactobacillus bacteria to be delivered on to the skin without being damaged by the soap. Lactobacillus and other strains of bacteria normally found in natural yoghurt, like bacteria including but not limited to Lactobacillus Acidophilus, Lactobacillus Casei, Lactobacillus Rhamnosus, Lactobacillus Salivarius, Bifidobacterium Bifidum, Bifidobacterium Lactis, Streptococcocus Thermophilus limit infections and cure pathology in the human body. In the present invention, these natural bacterial species found in yoghurt have been added to ordinary soap using a special gelatin and/or water soluble cellulose sandwich structure hence called ‘Gel Lactowich’, allowing the bacteria to survive in the soap and not be destroyed by the acids, alkalis and other chemical present in the soap. The bacteria are then easily transmitted to the user's skin when the soap is used with water. Once the beneficial bacteria as mentioned above are transmitted to the skin they immediately start curing infections from the skin. It prevents other infections from attacking the skin and keeps the skin healthy and infection free. The bacteria when delivered via the soap are able to provide cosmetic and health benefits to any part of the body where the soap is used. The problems solved by this Microbiotic Lactobacillus soap include, but is not limited to: athletes foot, smelly feet, body odour, dandruff, hair loss or fungal alopecia, vaginal candidiasis, vaginal thrush, ring worm, dry skin, scaly skin, bed sores of the elderly, atopic dermatitis, nail fungus of the hands and feet, itchy skin, removes wrinkles, makes skin younger and softer.

The ‘Gel Lactowich’ structure can be further modified into small pellets formed of an outer covering of gelatin and/or water soluble cellulose with live bacteria at its centre to form vesicles of gelatin and/or water soluble cellulose totally enclosing or encapsulating the bacteria at its core hence forth called ‘Gel Lactosphere’.

The soap can be used for delivering other chemically beneficial organic or inorganic substances apart from beneficial bacteria via the special gelatin and/or water soluble cellulose sandwich called ‘Gel Lactowich’ or the gelatin and/or water soluble cellulose ‘Gel Lactosphere's’.

The ‘Gel Lactowich’ and ‘Gel Lactosphere’ can be used in varying amounts and different designs in soap keeping the basic concept and structure of ‘Gel Lactowich’ and ‘Gel Lactosphere the same.

The invention will now be described by way of example and with references to the accompanying drawings in which:

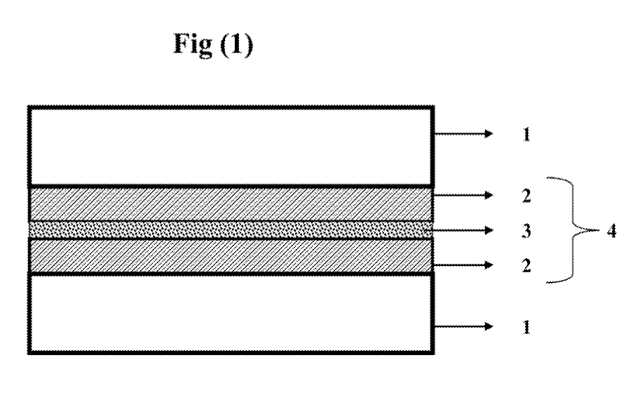

FIG. 1 shows a 2 dimensional side view of a bar of Microbiotic Soap with a Gel Lactowich structure in the middle of the soap having Lactobacillus bacteria at its centre.

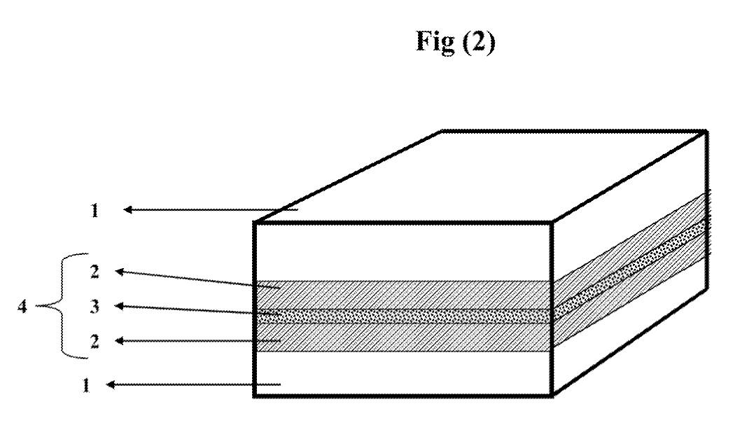

FIG. 2 shows a 3 dimensional diagrammatic front and side view of a bar of Microbiotic Soap with a Gel Lactowich structure in the middle of the soap having Lactobacillus bacteria at its centre.

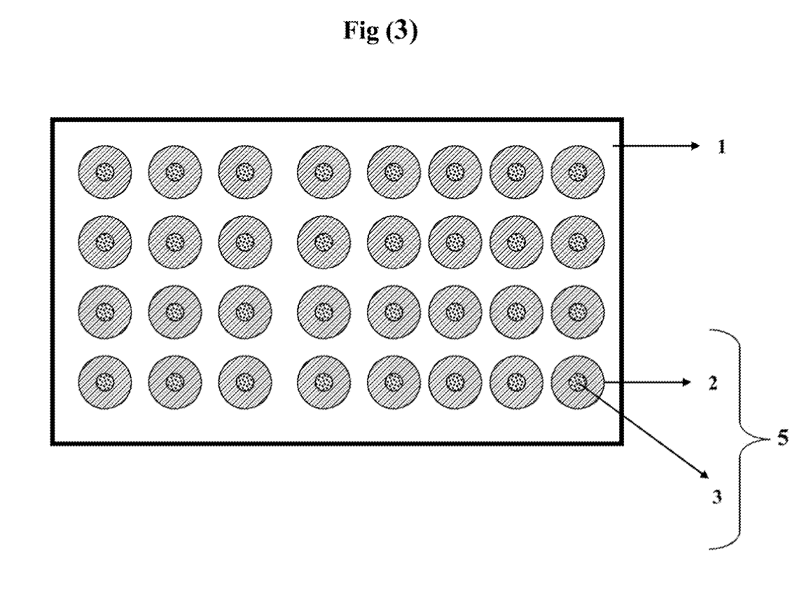

FIG. 3 shows a 2 dimensional side view of a bar of Microbiotic Soap having multiple Gel Lactospheres distributed through out the soap.

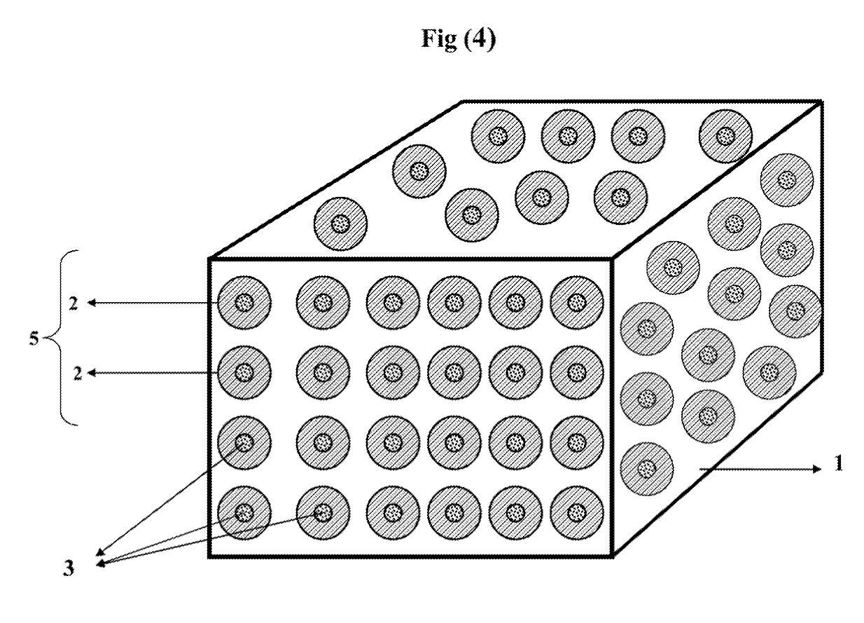

FIG. 4 shows a 3 dimensional diagrammatic view of a bar of Microbiotic Soap having multiple Gel Lactospheres distributed through out the soap.

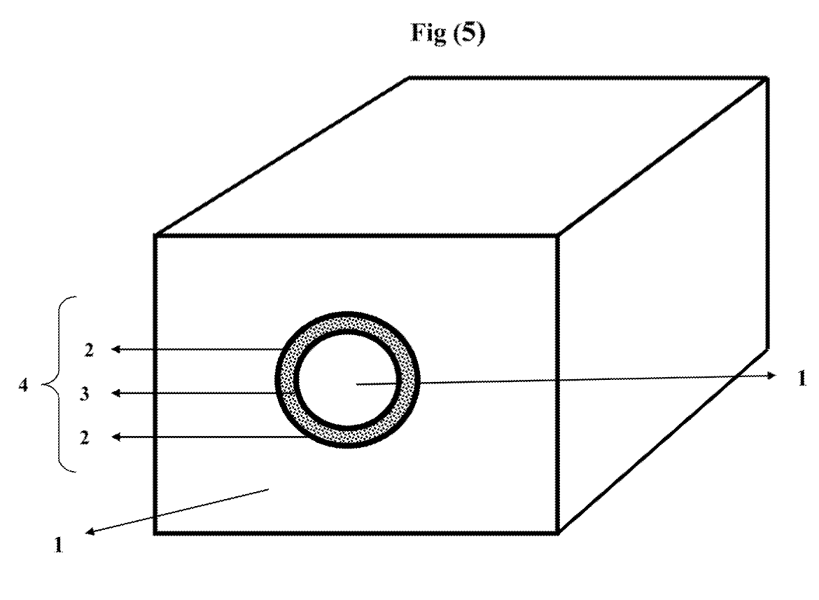

FIG. 5 shows a 3 dimensional diagrammatic view of a bar Microbiotic soap having an alternative/optional structure in which the Gel Lactowich is embedded in the centre of the soap as a ring through out the soap.

The details of FIGS. 1 and 2 show a bar of ordinary soap 1 used to make Microbiotic Soap which has at its centre two layers of gelatin and/or water soluble cellulose 2. The layers of the gelatin and/or water soluble cellulose have at its centre Lactobacillus species or other bacterial spores 3 as described previously. This structure together resembles a sandwich structure and is referred to as Gel Lactowich 4 in Microbiotic Soap. The Gel Lactowich 4 structure allows the bacterial spores to be stable while the soap is not in use. The outer layers of the Gel Lactowich 4, 2, prevent the harmful chemicals of the soap 1 from reaching the lactobacillus 3. When the soap is used in water, the water dissolves the soap 1 and Gel Lactowich 4 and the Lactobacillus 3 is freed from the structure and can deposit on the skin.

FIGS. 3 and 4 show a different version of the Microbiotoic Soap in which the soap 1, has Lactobacillus 3 surrounded by gelatin and/or water soluble cellulose 2 around a 360 degree angle like small spheres or balls with Lactobacillus 3 at it centre. This structure is called Gel Lactosphere 5 in Microbiotic soap. The Gel lactospheres 5 have lactobacillus 3 or other bacteria as mentioned before totally surrounded by gelatin and/or water soluble cellulose 2 and are embedded through out the soap. The Gel Lactospheres 5 keep the chemicals of the soap 1 from reaching the Lactobacillus 3 and thus protects them, keeping them alive. When the soap is used with water the Gel lactospheres 5 are gradually exposed to the water and the outer layer of gelatin and/or water soluble cellulose 2 from the Gel Lactospheres 5 melts away exposing the lactobacillus 3 which can then be delivered to the skin via the soap.

FIG. 5 is an optional diagram of a bar of Microbiotic Soap in which the Gel Lactowich 4 has been made into a ring structure resembling a pipe through out the centre of the Microbiotic Soap bar. The pipe has soap 1 at its centre and surrounding and the Gel Lactowhich 4 in between with Lactobacillus 3 in between the two layers of gelatin and/or water soluble cellulose 2.

1. A delivery system for alkali and acid sensitive beneficial Lactobacillus species bacteria from natural yoghurt and/or other beneficial bacteria on to human skin, comprising:
a solid soap bar and gelatin and/ or water soluble cellulose such that the bacteria are able to survive and reach human skin once the soap is used along with water for everyday hygiene purposes.

2. A delivery system according to claim 1 in which the alkali and acid sensitive Lactobacillus and other bacteria from natural yoghurt are inserted and sandwiched between two or more layers of gelatin and/or water soluble cellulose such that the acid and alkali from the soap is unable to reach and kill the bacteria owing to the gelatin and/or water soluble cellulose barrier called ‘Gel Lactowich’ allowing the delivery of live bacteria to human skin when the soap is used for hygiene purposes along with water.

3. A delivery system according to claim 1 in which the alkali and acid sensitive Lactobacillus and other bacteria from natural yoghurt are enclosed within gelatin and/or water soluble cellulose to form small round gelatin and/or water soluble cellulose encapsulated vesicles or spheres called ‘Gel Lactospheres’ in which the alkali and acid sensitive bacteria described before are totally sealed and protected such that the acid, alkali or other chemical from the soap is unable to reach and kill the bacteria owing to the gelatin and/or water soluble cellulose barrier allowing the delivery of live bacteria to human skin when the soap is used for hygiene purposes along with water. 4) The use of other chemical substance of organic or inorganic origin either in the gelatin and/or water soluble cellulose sandwich structure called ‘Gel Lactowich’ as outlined in claims 1 and 2 or in gelatin and/or water soluble cellulose vesicles called ‘Gel lactospheres’ as mentioned in claim 3 using a solid soap bar as a delivery system on to skin when the soap is used with water.
